(19) United States
(12) Patent Application Publication
(10) Pub. No.: US 2022/0379169 A1
(43) Pub. Date: Dec. 1, 2022

(54) System and Method for Real Time Machine Learning Model Training and Prediction Using Physiological Data

(71) Applicant: World Champ Tech LLC, Mountain View, CA (US)

(72) Inventor: James Alexander Mattis, Mountain View, CA (US)

(21) Appl. No.: 17/565,909

(22) Filed: Dec. 30, 2021

Related U.S. Application Data
(60) Provisional application No. 63/193,971, filed on May 27, 2021.

(51) Int. Cl. A63B 24/00; G06N 20/00

(52) U.S. Cl. CPC A63B 24/0062; G06N 20/00; A63B 2024/0065

(57) ABSTRACT

A computer-implemented method for machine learning and prediction relating to exercise by a user during a bout of exercise may include providing a physical activity measurement device that includes at least one input and at least one output; providing a machine learning model; via the at least one input, collecting physiological data from the user; adding the physiological data acquired by the collecting to the user's physiological data set; training the machine learning model on the user's said physiological data set; making at least one prediction for the user based on the application of the machine learning model to at least one input during the bout of exercise, and communicating at least one prediction to the user via at least one output.

This application claims priority to U.S. provisional patent application No. 63/193,971, filed May 27, 2021, which is hereby incorporated by reference in its entirety.

### FIELD OF THE INVENTION

The present invention relates to information technology systems and methods for measurement and evaluation thereof, and more specifically to machine learning and prediction systems and methods for use in exercise and training.

### BACKGROUND

Computer machine learning models rely on a two step process: first, data is used to train the model, and then second, the model is applied to new input data to make generalized predictions or classifications about the input data. Typically, these models are trained using large samples of data prior to deploying them in a production environment. Such model training may be performed on a dedicated computer before deploying the trained model data to smaller devices, such as mobile smart phones, or mobile smart watches for prediction and classification. Computer machine learning models include linear regression, logistic regression, cluster analysis, support vector machines, hidden Markov models, and artificial neural networks. Predictions by a computer machine learning model can be based on regression (which helps predict a quantity on a continuous scale), classification (which can predict discrete class labels), or both.

Such an approach to training a machine learning model may be problematic in exercise or fitness applications when attempting to classify an individual's exercise or fitness data to make predictions based on that data. Each individual will be slightly different from each other individual, so the use of a large sample of physiological data from a number of people, who may or may not be randomly selected, (the “physiological data set”) is unlikely to lead to unbiased predictions that apply specifically to a particular individual. For instance, if the physiological data set contains more men than women, applying a pre-trained model created using that physiological data set will produce biased results when used to make classifications or predictions about data collected from a woman. Such variables in a physiological data set that may result in bias are not limited to gender, may be subtle, and may not be screened for by a data collector. Further, a professional or semi-professional athlete will, by definition, have a physiology different from the average, such that a physiological data set aggregating data from a number of average people will lack relevance or usefulness relative to the prediction or classification of that athlete's health state. Additionally, since an individual's health and fitness is constantly in flux, there is only a low probability that a physiological data set corresponds precisely to the individual's current health and fitness state. This will lead to erroneous and biased predictions or classifications for the individual's current health and fitness. For example, a physiological data set that includes data from only a small percentage of asthmatic people may not be useful or even relevant to an individual with severe asthma.

Another problem with current machine learning models is their use of cloud based server systems to perform the machine learning model training and predictions. As an example, the IPHONE® smartphone of Apple Inc. of Cupertino, Calif. utilizes the SIRI® system to receive and carry out voice commands, and at least some processing of such voice commands typically is handed off from the phone to a data center, after which the data center transmits results back to the phone. Similar processing is performed by servers of Alphabet, Inc. of Mountain View, Calif. by smartphones using the ANDROID® operating system. The time it takes to transmit raw measurement data to the cloud based server, have the server perform the work to train the machine learning model based on this data, and return a prediction to the individual is limited by the speed and availability of the network and server, which limits the ability of a cloud based approach to provide real-time predictions or classifications. Cloud based approaches are only as reliable as the network they use for data transmission, and there are many remote locations around the world where outdoor physical activity might be enjoyed with limited, or no network availability. For example, hiking in even slightly hilly or mountainous areas not far from civilization may occur in areas with spotty, or completely absent wireless service. The use of a wireless internet service such as STARLINK® wireless of Space Exploration Technologies Corp. of Hawthorne, Calif. may overcome the problem of a complete lack of network availability, but it exacerbates the problem of network latency and transmission time, because every signal must be transmitted several hundred miles into the air, to one or more other STARLINK® satellites, and then back down several hundred miles to the surface of the Earth. Thus, cloud-based approaches to machine learning models necessarily take longer to provide usable results to a user, due to the communication times involved.

Additionally, cloud server approaches face significant data security concerns when transmitting personal health data over a network—even when the data is encrypted during transmission. In the United States, the Health Insurance Portability and Accountability Act of 1996 (HIPAA) imposes strict requirements on health data privacy, and the data is vulnerable to attack when it is unencrypted on the cloud server for training the machine learning model or making predictions from the trained model.

Therefore, there is a need in the art of mobile or wearable technology to provide a health monitoring system that overcomes these limitations.

### SUMMARY OF THE INVENTION

According to some aspects of the invention, a physical activity measurement device may include a processing unit; at least one input connected to the processing unit, that at least one input related to exercise by a user; at least one output connected to the processing unit; at least one storage device; a machine learning model stored in the at least one storage device; where the machine learning model utilizes the at least one input to train itself and to make one or more exercise-related predictions to the user.

According to some aspects of the invention, a computer-implemented method for machine learning and prediction relating to exercise by a user during a bout of exercise may include providing a physical activity measurement device that includes at least one input and at least one output; providing a machine learning model; via the at least one input, collecting physiological data from the user; adding the physiological data acquired by the collecting to the user's physiological data set; training the machine learning model on the user's physiological data set; and making at least one prediction for the user based on the application of the machine learning model to at least one input during the bout of exercise.

According to some aspects of the invention, a computer-readable medium configures a processing unit of a user's physical activity measurement device, which includes at least one input and at least one output in communication with the processing unit, to perform collecting physiological data from the user, via the at least one input; adding the collected user physiological data to a user's physiological data set; training a machine learning model on the user's physiological data set; and making at least one prediction for the user based on the application of the machine learning model to at least one input during the bout of exercise.

According to some aspects of the invention, a computer system incorporates a range of measurement and computing elements in a novel configuration, and a software program may run on this system to transform raw input measurement data into a trained machine learning model, and make predictions or classifications from the model. This invention improves on the existing art by enabling individuals to have accurate, real-time predictions or classifications based on their own physical activity data during an exercise activity, without concerns of data security or network access. The real time predictions or classifications provided by this invention allow individuals to precisely manage and tune an exercise workout plan during the workout, where such management and tuning can help maximize fitness improvements while minimizing the chance of fatigue, burnout, or injury.

According to some aspects of the present invention, it seeks to address some of the issues raised in the Background section by providing a portable system capable of performing real time training of a machine learning model from input data streams or files of measured human physical activity sensor data, combined with an algorithm or method for training the model based on the input data, and an algorithm or method for making predictions or classifications from the trained model and input data. The solution also requires a mechanism for communicating the prediction or classification results to the user.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

FIG. 1 is a schematic view of an exemplary human physical activity measurement device.

FIG. 2 is a schematic view of a bicycle having at least one sensor associated therewith.

FIG. 3 is a flowchart of a machine learning and prediction process.

The use of the same reference symbols in different figures indicates similar or identical items.

### DETAILED DESCRIPTION

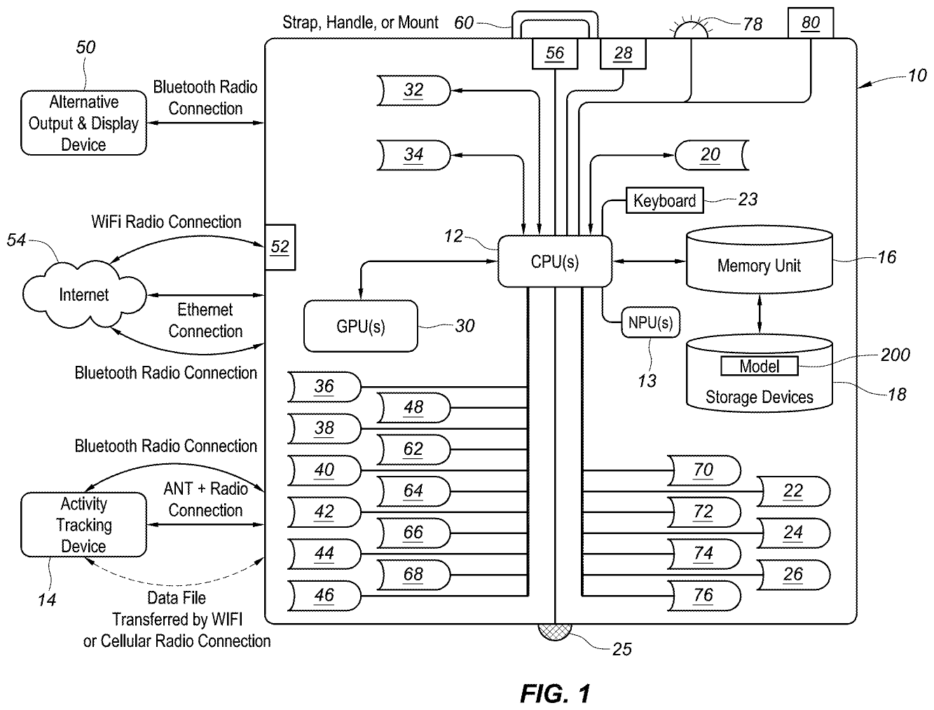

Referring to FIG. 1, an exemplary human physical activity measurement device 10 is shown. The physical activity measurement device 10 may be attached to a user. Such attachment may be direct or indirect. Direct attachment may be performed by adhesive, one or more elastic bands, or by any other suitable manner. Indirect attachment may be performed by attachment to a user's clothes, athletic protective gear, shoes, or any other suitable attachment or system. Indirect attachment instead may be performed by the attachment of a remote sensor or sensors 14 to the user, where the sensor or sensors 14 are connected for the transmission of data through a wire or wirelessly, such as by the BLUETOOTH® protocol of the Bluetooth SIG of Kirkland, Wash. The physical activity measurement device 10 may be a smartphone, a tablet computer, a wearable computer, or any other suitable device. The sensor or sensor 14 may be, or may be included in, a smartwatch, an activity tracking device such as a FITBIT® activity tracking device of Alphabet, Inc. of Mountain View, Calif., a smartring such as the OURA® smartring of Ōura Health Oy of Oulu, Finland, or any other suitable device.

The physical activity measurement device 10 may include a central processing unit (CPU) 12. The physical activity measurement device 10 may include a memory unit 16 connected to the CPU 12. According to some embodiments, the memory unit 16 may be, and/or may include, solid state memory such as random access memory (RAM). The physical activity measurement device 10 may include one or more storage devices 18 connected to the memory unit 16. According to some embodiments, the one or more storage devices may be, and/or may include, solid state memory such as random access memory (RAM). According to other embodiments, the one or more storage devices 18 may be, and/or may include, volatile and/or nonvolatile memory, removable and/or non-removable media such as a hard drive, read-only memory (ROM), electrically erasable programmable read-only memory (EEPROM), flash memory or other memory technology, other magnetic storage devices, or any other medium which can be used to store the desired information and which can accessed by an instruction execution system implemented in any method or technology for storage of information such as computer readable instructions, data structures, program modules or other data.

The one or more storage devices 18 may store a machine learning model 200 that implements functionality of the invention, as described in greater detail below. According to some embodiments, the one or more storage devices 18 may provide long-term storage of the machine learning model 200 and/or other data, and the memory unit 16 may provide short-term storage of some or all of the machine learning model 200 and/or other data in a location and/or medium that is more quickly accessible to the CPU 12.

The physical activity measurement device 10 may include, or otherwise be associated with, one or more input and/or output units. An “input” is any device that acquires data from the user and/or the environment, and an “output” is any device that communicates information to the user. As one example of an input unit, the physical activity measurement device 10 may include a touchscreen 20, where the user may enter input using that touchscreen 20. The touchscreen 20 may be electronically connected to the CPU 12 directly or indirectly. Devices such as the touchscreen 20 may be both inputs and outputs. As another example, the physical activity measurement device 10 may include a graphical display device 22. The graphical display device 22 may be electronically connected to the CPU 12 directly or indirectly. According to some embodiments, the touchscreen 20 and the graphical display device 22 may be the same unit, such as on a conventional smartphone or tablet computer. According to other embodiments, the touchscreen 20 may be different from and separate from the graphical display device 22. For example, where the user wears an activity tracking device 14 that includes a screen, the touchscreen 20 may be considered to be the screen of the activity tracking device 14, and the graphical display device 22 may be considered to be the screen of a smartphone associated with the activity tracking device 14.

Optionally, the physical activity measurement device 10 may include or be connectable to a keyboard 23, which the user may utilize to enter input.

Optionally, the physical activity measurement device 10 may include or be connectable to a microphone 25, which the user may utilize to enter voice input.

As another example of an output unit, the physical activity measurement device 10 may include one or more audio output devices 24, such as one or more speakers. The audio output device or devices 24 may be electronically connected to the CPU 12 directly or indirectly. As another example of an output unit, the physical activity measurement device 10 may include a mechanical vibration device 26. The mechanical vibration device 26 provides for haptic as well as gentle audible output, whether instead of or in addition to the audio output from the audio output device 24. The mechanical vibration device 26 may be electronically connected to the CPU 12 directly or indirectly.

The physical activity measurement device 10 may include a graphics processor unit (GPU) 30 that may be useful in assisting the CPU 12 in rendering output for display on the touchscreen 20 and/or graphical display device 22. Further, a machine learning model 200 (described below) may run in whole or in part on the GPU 30. For example, on some smartphones such as the IPHONE® smartphones of Apple, Inc. of Cupertino, Calif., calculations relating to matrix mathematics may be performed at least in part by the GPU 30. The physical activity measurement device 10 may include a neural processing unit (NPU) 13 on which a machine learning model 200 (described below) may run in whole or in part. For example, some models of IPHONE® smartphones of Apple, Inc. of Cupertino, Calif. include a neural processing unit 13 that is an A11 Bionic processor specifically intended to perform machine learning in an energy-efficient manner.

The physical activity measurement device 10 may include a radio transceiver 32 such as a BLUETOOTH® radio that utilizes the BLUETOOTH® protocol of the Bluetooth SIG of Kirkland, Wash. According to other embodiments, the radio transceiver 32 may be an ANT+™ radio that utilizes the ANT+™ protocol of ANT Wireless, a division of Garmin Canada, of Cochrane, Alberta, Canada. Any other radio transceiver 32 and/or protocol may be used that is suitable for local communication without the use of the Internet 54 or other data network; the description of the wireless protocols above is merely exemplary and not limiting. As used in this document, the terms “local communication” and “locally” refer to a distance of less than substantially 100 meters. 100 meters is substantially the range of the ANT+ radio. According to other embodiments, the wireless protocol utilized is suitable for personal communication. As used in this document, the terms “personal communication” and “personally” refer to a single, limited area in proximity to the user, within the user's reach. Devices such as the physical activity measurement device 10, a smartphone, a tablet computer, a wearable computer, a smartwatch, an activity tracking device, or a smartring are often on the user's person, or within reach, when the user is utilizing them in the course of a bout of exercise and/or while training of the machine learning model 200 is performed.

Optionally, the physical activity measurement device 10 may include a USB port 28 or other port through which one or more additional input and/or output devices 50 may be connected to the physical activity measurement device 10 and to the CPU 12. As one example, a keyboard may be connected to the USB port 28. As another example, one or more speakers may be connected to the USB port 28. Optionally, one or more additional input and/or output devices 50 may be connected to the physical activity measurement device 10 wirelessly through the radio transceiver 32, such as by the BLUETOOTH® protocol of the Bluetooth SIG of Kirkland, Wash., the ANT+™ protocol of ANT Wireless, a division of Garmin Canada, of Cochrane, Alberta, Canada, or any other suitable wireless communication protocol.

According to some embodiments, the physical activity measurement device 10 may be connectable to the Internet 54. Such a connection may be accomplished wirelessly through the radio transceiver 32, such as by the BLUETOOTH® protocol of the Bluetooth SIG of Kirkland, Wash. According to other embodiments, such a connection may be accomplished wirelessly through a WI-FI® transceiver 52, using the WI-FI® protocol of the Wi-Fi Alliance of Austin, Tex. According to other embodiments, such a connection may be accomplished by a wired Ethernet connection, such as through the USB port 28 or a separate Ethernet port 56 associated with the physical activity measurement device 10.

According to some embodiments, at least one of the input and/or output units may be included in the activity tracking device 14 instead of, or in addition to, the physical activity measurement device 10.

The physical activity measurement device 10 may include, or otherwise be associated with, one or more sensors of different types. Each of these sensors is an input, as described above. As one example of a sensor, the physical activity measurement device 10 may include a Global Positioning System (GPS) transceiver 34. The GPS transceiver 34 may be electronically connected to the CPU 12 and/or other processing units of the physical activity measurement device 10 directly or indirectly. According to some embodiments, the GPS transceiver 34 is a true transceiver that both receives and decodes location signals, on the one hand, and may transmit the location of the user automatically or in response to a user request, on the other hand. In this way, if a user is hiking and becomes lost, the user can indicate his or her location to search and rescue personnel. Further, if the user experiences physical distress during exercise, the user can indicate his or her location to emergency services such as an ambulance. According to other embodiments, the GPS transceiver 34 is a receiver that receives and decodes location signals, but does not transmit the location of the user. As another example of a sensor, the physical activity measurement device 10 may include one or more accelerometers 36. The one or more accelerometers 36 may be electronically connected to the CPU 12 and/or other processing units of the physical activity measurement device 10 directly or indirectly. Advantageously, three or more accelerometers 36 may be utilized, in order to measure the accelerations applied to the physical activity measurement device 10 in three orthogonal directions, and in additional directions if desired. As another example of a sensor, the physical activity measurement device 10 may include one or more gyroscopes 38. The one or more gyroscopes 38 may be electronically connected to the CPU 12 and/or other processing units of the physical activity measurement device 10 directly or indirectly. Advantageously, three or more gyroscopes 38 may be utilized, in order to measure the accelerations applied to the physical activity measurement device 10 in three orthogonal directions, and in additional directions if desired. The gyroscopes 38 may be conventional gyroscopes, MEMS gyroscopes, vibrating structure gyroscopes (CVGs), or any other suitable type gyroscope. As another example of a sensor, the physical activity measurement device 10 may include a heart rate sensor 40. The heart rate sensor 40 may be electronically connected to the CPU 12 and/or other processing units of the physical activity measurement device 10 directly or indirectly.

Optionally, the physical activity measurement device 10 may include a pulse oximeter 42. The heart rate sensor 40 may be electronically connected to the CPU 12 and/or other processing units of the physical activity measurement device 10 directly or indirectly.

Optionally, the physical activity measurement device 10 may include at least one thermometer 44. The thermometer 44 may be electronically connected to the CPU 12 and/or other processing units of the physical activity measurement device 10 directly or indirectly. At least one thermometer 44 may measure the body temperature of the user. Optionally, at least one thermometer 44 may measure the temperature of the ambient air.

Optionally, the physical activity measurement device 10 may include a blood pressure sensor 46. The blood pressure sensor 46 may be electronically connected to the CPU 12 and/or other processing units of the physical activity measurement device 10 directly or indirectly.

Optionally, the physical activity measurement device 10 may include a respiration rate sensor 48. The respiration rate sensor 48 may be electronically connected to the CPU 12 and/or other processing units of the physical activity measurement device 10 directly or indirectly.

Optionally, the physical activity measurement device 10 may include a blood lactate sensor 62. The blood lactate sensor 62 may be electronically connected to the CPU 12 and/or other processing units of the physical activity measurement device 10 directly or indirectly. Advantageously, the blood lactate sensor 62 is noninvasive and measures blood lactate in real time. Alternately, the blood lactate sensor 62 requires a drop of blood to be placed in contact therewith when blood lactate is to be measured.

Optionally, the physical activity measurement device 10 may include an altimeter 64, because altitude is a convenient proxy for concentration of ambient oxygen. The altimeter 64 may be electronically connected to the CPU 12 and/or other processing units of the physical activity measurement device 10 directly or indirectly. Alternately, the altitude of a given hike, run, bike ride or other activity may be determined using an electronic terrain map in combination with location data as determined from the GPS transceiver 34. If the latitude and longitude of the user is known, the altitude of the user can be determined from an electronic terrain map such as the Google Maps product of Alphabet, Inc. of Mountain View, Calif. However, a built-in altimeter 64 provides the advantages of not requiring a connection to the Internet 54 to determine altitude based on real-time position, and not requiring additional storage in the storage device 18 for storing a terrain map within the physical activity measurement device 10.

Optionally, the physical activity measurement device 10 may include a vibration sensor 66. The vibration sensor 66 may be electronically connected to the CPU 12 and/or other processing units of the physical activity measurement device 10 directly or indirectly. As one example, the vibration sensor 66 may be an HVM200 of PCB Piezoelectronics of Depew, N.Y.

Optionally, the physical activity measurement device 10 may include a blood glucose sensor 68. The blood glucose sensor 68 may be electronically connected to the CPU 12 and/or other processing units of the physical activity measurement device 10 directly or indirectly.

Optionally, the physical activity measurement device 10 may include a radar transceiver 70. The radar transceiver 70 may be electronically connected to the CPU 12 and/or other processing units of the physical activity measurement device 10 directly or indirectly. The radar transceiver 70 may be useful in measuring one or more vital signs of the user, such as heart rate and respiratory rate, in a noninvasive manner. The radar transceiver 70 may be, for example, the FDA-approved XK300™ sensor of Xandar Kardian of Toronto, Ontario, Canada.

Optionally, the physical activity measurement device 10 may include a sonar transceiver 72. The sonar transceiver 72 may be electronically connected to the CPU 12 and/or other processing units of the physical activity measurement device 10 directly or indirectly.

Optionally, the physical activity measurement device 10 may include, or be connectable to, a weight sensor 74 such as a scale. The weight sensor 74 may be electronically connected to the CPU 12 and/or other processing units of the physical activity measurement device 10 directly or indirectly. According to other embodiments, the weight sensor 74 may be omitted, and the user manually inputs weight measurements to the physical activity measurement device 10 via the touchscreen 20 or other input, before and/or after the bout of exercise.

Optionally, the physical activity measurement device 10 may include, or be connectable to, a clock 76. The clock 76 may include a timer and/or stopwatch function as well. The clock 76 may be electronically connected to the CPU 12 and/or other processing units of the physical activity measurement device 10 directly or indirectly.

Optionally, the physical activity measurement device 10 may include, or be connectable to, a camera 80. The camera 80 may be electronically connected to the CPU 12 and/or other processing units of the physical activity measurement device 10 directly or indirectly. The camera 80 may be used in addition to, or instead of, sensors such as the radar transceiver 70 and the sonar transceiver 72 to capture motion data relating to the user's motion during a bout of exercise.

According to some embodiments, at least one of the sensors may be included in the activity tracking device 14 instead of, or in addition to, the physical activity measurement device 10.

As shown in FIG. 1, several groups of sensors and/or input devices are connected to the CPU 12 via a bus connection. FIG. 1 is not limiting as to the type of connection between each sensor or input device on the one hand and the CPU 12 on the other; rather, FIG. 1 illustrates a bus connection for ease of visualization of the invention. It will be apparent to those skilled in the art that two or more sensors may be connected to the CPU 12 in series or in parallel, may be connected to the CPU 12 via a bus that may be multiplexed or may not be multiplexed, may be connected individually to the CPU 12, or may be connected to the CPU 12 in any other suitable manner.

Where the physical activity measurement device 10 is a wearable device, the physical activity measurement device 10 optionally may include at least one strap 60 or other mount that is configured to allow the user to wear the physical activity measurement device 10 securely and conveniently.

Utilizing its hardware components as described above, the physical activity measurement device 10 may perform one or more measurements. The physical activity measurement device 10 may output such measurements as an electronic data stream that includes individual measurements as they are taken by the device, and/or an electronic data file that includes a set of measurements taken by the device over a period of time. Further, some measurements are calculated by a processing unit such as the CPU 12, the GPU 30 and/or the NPU 13 based on raw data obtained from the sensors connected to the CPU 12. It will be understood by those skilled in the art that computation performed within the physical activity measurement device 10 may be performed by one or more processing units therewithin, notwithstanding the nomenclature used to describe specific processing units. Further, it will also be understood by those skilled in the art that some or all of such computation may be performed by processing units in devices local to the user, where the physical activity measurement device 10 transmits data to and receives data from such local devices. Below, a list of exemplary measurements is provided by way of example and not limitation. The physical activity measurement device 10 need not perform each and every one of such measurements, nor are such measurements listed below limiting as to the type and quantity of measurements that may be performed by the physical activity measurement device 10.

One such exemplary measurement is energy produced by the user during a particular time period.

Another such exemplary measurement is power produced by the user at various points during a time period, along with the time difference between each power measurement. The different points in time at which power is determined may be at regular intervals, or may be at varying intervals.

Another such exemplary measurement is the volumetric flow rate of nitrogen, oxygen, and carbon dioxide exhaled by the user at various measurement points during a time period, and the length of time between each measurement point. The respiration rate sensor 48 may be configured to sense the components of the user's exhaled breath, such that the respiration rate sensor 48 can measure the volumetric flow rate of one or more individual gases in the user's exhaled breath. Alternately, one or more additional sensors may be associated with the respiration rate sensor 48, or may be provided separately from the respiration rate sensor 48, such that those sensors can measure the volumetric flow rate of one or more individual gases in the user's exhaled breath.

Another such exemplary measurement is the blood lactate level of the user at various measurement points during a time period, and the length of time between each measurement point.

Another such exemplary measurement is the blood glucose level of the user at various measurement points during a time period, and the length of time between each measurement point.

Another such exemplary measurement is the weight and/or mass of the user at the beginning of the bout of exercise, at the end of the bout of exercise, and/or at various measurement points during the time of a bout of exercise, and the length of time between each measurement point.

Another such exemplary measurement is the time, such as the instantaneous time, the time of the beginning of the bout of exercise, the time elapsed since the start of the bout of exercise, and the time of the ending of the bout of exercise.

Another such exemplary measurement is the instantaneous heart rate of the user at various measurement points during a time period, and the times when each measurement was taken.

Another such exemplary measurement is the acceleration of the user at various points during a time period, measured by a set of accelerometers 36 attached to the user and oriented to measure acceleration in three orthogonal directions, along with the times when each set of measurements was taken.

Another such exemplary measurement is the angular acceleration of the user at various points during a time period measured by a set of gyroscopes 38 attached to the user and oriented to measure angular acceleration in three orthogonal directions, along with the times when each set of measurements was taken.

Another such exemplary measurement is the latitude, longitude, and altitude of the user measured at various point during a time period, along with the times when each measurement was taken.

Another such exemplary measurement is the change in body temperature of a user measured at various points during a time period, along with the times when each measurement was taken. The ambient air temperature, and the change in ambient air temperature, optionally may be measured at various points during a time period, along with the times when each measurement was taken.

Another such exemplary measurement is the velocity of the user at various points during a time period, along with the times when each measurement was taken. That velocity may be determined by the change in position of the user over time, where that position may be determined via GPS data received by the GPS transceiver 34.

Another such exemplary measurement is the gradient of the surface along which the user is traveling, such as by running, walking or bicycling, at various points during a time period, along with the times when each measurement was taken. That gradient may be determined by the change in altitude of the user over time, where that altitude may be determined by the altimeter 64. Alternately, that gradient may be determined via GPS data received by the GPS transceiver 34 in combination with an electronic terrain map that includes the altitudes of terrain associated with latitude/longitude coordinates.

Another such exemplary measurement is one or more properties of the physical media through which the user is moving (such as water temperature, water salinity, and water velocity relative to the user for swimming; air temperature, air relative humidity, user elevation above sea level for running, walking, hiking, skiing, bicycling, and other motion based activities), measured at various points during a time period, along with the times when each measurement was taken. One or more of such measurements may be performed with sensors specifically configured to measure such properties. Alternately, one or more of such measurements may be performed by utilizing the connection to the Internet 54 to query external servers that provide weather data (such as air relative humidity) for a particular location, where the Internet 54 is available at that location.

Another such exemplary measurement or measurements are personal data relating to the user, such as age, gender, and/or weight. The user may enter this information such as via the touchscreen 20 or microphone 25. The user optionally may enter a birthday rather than an age, and the user's age can be calculated each time the user exercises based on that birthday. The user may enter a value for weight at the beginning and/or end of a bout of exercise, or at another time.

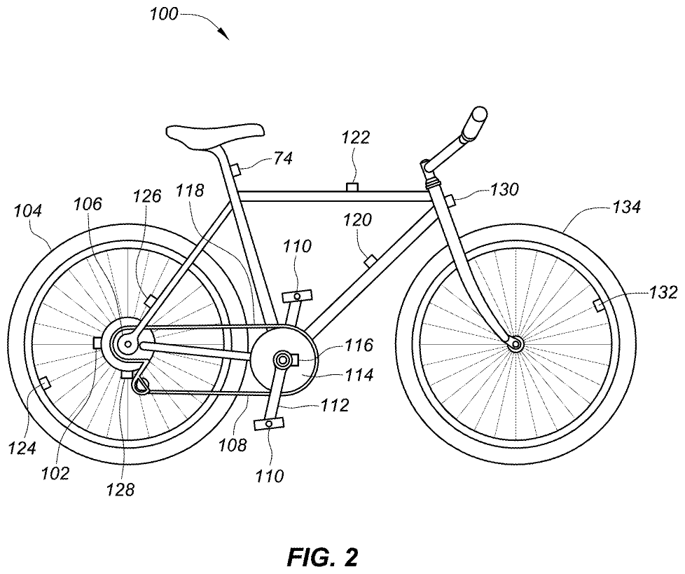

One or more sensors may be connected to or otherwise associated with exercise equipment that may be utilized by a user. As one example, referring also to FIG. 2, a user's bicycle 100 may be outfitted with one or more sensors that are usable when the user is riding the bicycle 100. At least one torque sensor 102 may be associated with the sprocket 106 of the rear wheel 104 of the bicycle 100. The torque sensor 102 has a data connection with the physical activity measurement device 10, such as a BLUETOOTH® connection with the radio transceiver 32. A chain 108 connects the sprocket 106 to a chainring 114. The chainring 114 is connected to crank arms 112, each of which is connected to a pedal 110. At least one torque sensor 116 may be associated with the chainring 114. The torque sensor 114 has a data connection with the physical activity measurement device 10, such as a BLUETOOTH® connection with the radio transceiver 32. A vibration sensor 120 may be connected to the bicycle 100 at any suitable location on the bicycle 100. The vibration sensor 120 has a data connection with the physical activity measurement device 10, such as a BLUETOOTH® connection with the radio transceiver 32. One or more accelerometers 122 may be connected to the bicycle 100 at any suitable location on the bicycle 100. The at least one accelerometer 122 has a data connection with the physical activity measurement device 10, such as a BLUETOOTH® connection with the radio transceiver 32. One or more gyroscopes 124 may be connected to the bicycle 100 at any suitable location on the bicycle 100. The at least one gyroscope 124 has a data connection with the physical activity measurement device 10, such as a BLUETOOTH® connection with the radio transceiver 32. One or more weight sensors 74 may be connected to the bicycle 100 at any suitable location on the bicycle 100. The at least one weight sensor 74 has a data connection with the physical activity measurement device 10, such as a BLUETOOTH® connection with the radio transceiver 32. One or more strain gauges 126 may be connected to the bicycle 100 at any suitable location on the bicycle 100. The at least one strain gauge 126 has a data connection with the physical activity measurement device 10, such as a BLUETOOTH® connection with the radio transceiver 32. One or more force sensors 128 may be connected to the bicycle 100 at any suitable location on the bicycle 100. The at least one force sensor 128 has a data connection with the physical activity measurement device 10, such as a BLUETOOTH® connection with the radio transceiver 32. A force sensor 128 is a transducer that converts an input mechanical load, weight, tension, compression or pressure into an electrical output signal. One or more velocity sensors 130 may be connected to the bicycle 100 at any suitable location on the bicycle 100. The at least one velocity sensor 130 has a data connection with the physical activity measurement device 10, such as a BLUETOOTH® connection with the radio transceiver 32. One or more angular velocity sensors 132 may be connected to the bicycle 100 at any suitable location on the bicycle 100. The at least one angular velocity sensor 132 has a data connection with the physical activity measurement device 10, such as a BLUETOOTH® connection with the radio transceiver 32.

An exemplary measurement performed by the bicycle 100 may be the torque applied to the chainring 114 by the application of force by the user to the pedals 110, and the linear force applied to the upper span 118 of the chain 108, and the times when each measurement of force and velocity was taken. That torque may be measured by the torque sensor 116. For example, the linear force applied to a bicycle chain 108 may be determined by the mathematical relationship: torque=force×chainring radius; thus, it follows that the linear force applied to the bicycle chain 108 at any given moment is equal to the torque measured by the torque sensor 116 at that moment, divided by the known, fixed diameter of the chainring 114. The radius of the chainring 114 is fixed, and the user can enter that radius into the physical activity measurement device 10, where that radius can be stored until deleted by the user. Optionally, the user can enter the radius of the chainring 114 of more than one bicycle 100 into the physical activity measurement device 10, because serious competitive bicyclists typically own and train on multiple different bicycles 100. The velocity of the chain 108 at a particular moment also can be calculated based on the rotational speed of the chainring 114, which may be sensed by the torque sensor 116.

Another exemplary measurement performed by the bicycle 100 may be torque applied to the rear wheel 104 of the bicycle, and the times when each measurement of torque was taken. That torque may be measured by a torque sensor 102 associated with the sprocket 106 of the rear wheel 104. The measurement of the torque force actually received at the sprocket 106 at a particular moment, divided by the measurement of the torque applied to the chainring at the same moment, is a measurement of efficiency at which the bicycle 100 is being powered by the user.

Another exemplary measurement performed by the bicycle 100 may be vibration of the bicycle 100, and the times when each measurement of vibration was taken. The vibration of the bicycle 100 may be measured by the vibration sensor 120.

Another exemplary measurement performed by the bicycle 100 may be the velocity of the bicycle 100. Acceleration of the bicycle 100 can be determined with the velocity measurement performed by the one or more velocity sensors 130 and the time measurement performed by the clock 76.

Another exemplary measurement performed by the bicycle 100 may be the velocity of the bicycle 100. Distance traveled by the bicycle 100 can be determined with the angular velocity measurement performed by the angular velocity sensors 132, where the radius of the rear wheel 104 or front wheel 136 of the bicycle 100 at which angular velocity is measured is known by the machine learning model 200.

Another exemplary measurement performed by the bicycle 100 may be acceleration of the bicycle 100, and the times when each measurement of acceleration was taken. The acceleration of the bicycle 100 may be measured by the at least one accelerometer 122. The bicycle 100 has a known mass, and the force applied to the rear wheel 104 can be determined as described above, so the measurement of acceleration may be calculated using the relationship force=mass×acceleration, such that acceleration=force/mass. The mass of a bicycle 100 operated by a user is substantially constant across time, so the user can weigh the bicycle 100 at a point in time and enter that mass into the physical activity measurement device 10, where that mass can be stored until deleted by the user. Optionally, the user can enter the mass of more than one bicycle 100 into the physical activity measurement device 10, because serious competitive bicyclists typically own and train on multiple different bicycles 100.

Another exemplary measurement performed by the bicycle 100 may be angular acceleration of the rear wheel 104 of the bicycle 100, and the times when each measurement of angular acceleration was taken. The angular acceleration of the rear wheel 104 of the bicycle 100 may be measured directly by the at least one gyroscope 124 mounted to the rear wheel 104.

Another exemplary measurement performed by the bicycle 100 may be measurement of the weight of the user during a ride. The weight measurement may be performed intermittently, such as once per minute or once per five minutes, or may be measured generally constantly. The measurement of weight during exercise can provide information about weight loss, and also about the amount of water both consumed by and sweated away from the user.

Other exercise equipment may be configured with sensors in a similar manner to the bicycle 100 and its sensors described above, and such sensors may perform measurements in a similar manner to that described above. Such other exercise equipment includes, but is not limited to, treadmills, wheelchairs, stair climbers, rowing machines, and ski machines.

Method

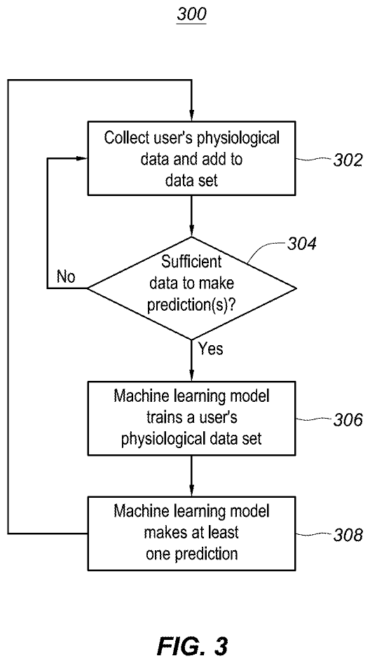

Referring to FIG. 3, a computer-implemented method 300 is shown. As described above, the use of a physiological data set derived from a large group of people is unlikely to lead to unbiased predictions that apply specifically to a particular individual. The method 300 utilizes the user's own physiological data to train a machine learning model 200, and does so on the user's own device rather than in the cloud or otherwise remotely.

In box 302, the user's physiological data is acquired and added to the user's physiological data set. Such data is collected from one or more of the sensors described above, and stored in the memory unit 16 and/or storage device 8 described above. According to some embodiments, the user's physiological data is acquired during a bout of exercise, after which data collection ceases until the next bout of exercise. The user may provide input to the machine learning model 200 such as via the touchscreen 20 of the physical activity measurement device 10 to indicate that the bout of exercise is beginning, and also may provide input to the machine learning model 200 via the physical activity measurement device 10 that the bout of exercise has ended. According to other embodiments, in box 302 the physical activity measurement device 10 senses that the bout of exercise has ended by sensing a lowered heartrate and/or other sensed data. According to other embodiments, the physical activity measurement device 10 continuously collects data from the user from at least one sensor. For example, where the user constantly wears the physical activity measurement device 10, the physical activity measurement device 10 may continuously collect heart rate data, respiration data, and blood oxygen level data from the user. The data collected from the user in box 302 is added to and stored as part of the user's physiological data set.

In box 304, the method 300 determines whether the user's physiological data set is sufficiently large to enable the machine learning model to make useful predictions. This determination may be made in several ways. As one example, the user's physiological data set may be considered to be sufficiently large with respect to a particular measurement after a specific number of that measurement has been collected. For example, for heart rate data, the user's physiological data set may be sufficiently large after 1000 measurements of heart rate data have been collected. As another example, the user's physiological data set may be considered to be sufficiently large with respect to a particular measurement after a particular time across which that measurement has been collected. For example, for power data, the user's physiological data set may be sufficiently large after 20 minutes of power data has been collected. It can be seen from the examples of this paragraph that the user's physiological data set may be sufficiently large with regard to some measurements and thus able to make predictions based on those measurements, while simultaneously the user's physiological data set may not be sufficiently large with regard to other measurements and thus unable to make predictions based on those other measurements.

According to other embodiments, the determination is a simple count of bouts of exercise. The physical activity measurement device 10 stores the number of bouts of exercise that it has monitored, which equals the number of times that box 302 has been performed. When that number reaches a threshold level that is preset and stored in the physical activity measurement device 10, then box 304 determines that sufficient data has been collected. According to other embodiments, the determination whether the user's physiological data set is sufficiently large to enable the machine learning model to make useful predictions is made based on the volume of data collected, not on the number of bouts of exercise performed by the user. In this way, the overall duration of exercise, not the number of bouts of exercise, may be determinative of the sufficiency of the user's physiological data set. When that duration of data reaches a threshold level that is preset and stored in the physical activity measurement device 10, then box 304 determines that sufficient data has been collected. According to other embodiments, at box 304 any other suitable criteria may be applied to evaluate whether the user's physiological data set is sufficiently large to enable the machine learning model to make useful predictions.

Next, at box 306, the machine learning model 200 trains on the user's physiological data set. Training a machine learning model 200 is known to those skilled in the art. In brief, the process of training the machine learning model 200 includes providing the machine learning model 200 with training data to learn from. The machine learning model 200 finds patterns in the user's physiological data set that map the input data attributes to the predictions to be made by the machine learning model 200. The machine learning model 200 runs locally, and is trained locally, on the physical activity measurement device 10. According to some embodiments, the machine learning model 200 may be, or may be part of, an app that runs on a user's smartphone, tablet or smart watch. Machine learning models, and training of machine learning models, are known in the art and may be implemented by one skilled in the art. A particular machine learning model that may be implemented as the machine learning model 200 may be a logistic regression model, a linear regression model, a nonlinear regression model, a deep neural network, convolutional network, an unsupervised learning technique (e.g., clustering), a Bayesian network, a hidden Markov model, a long short-term memory (LSTM), a fully recurrent neural network (RNN), a recursive neural network, a Hopfield network, an echo state network, a bi-directional RNN, a continuous-time RNN, a hierarchical RNN, a recurrent multilayer perceptron, a second order RNN, a multiple timescales RNN, a bidirectional associative memory, a convolutional neural network, a neural history compressor, a deep belief network, a convolutional deep belief network, a large memory storage and retrieval neural network, or other form of machine learning model. Advantageously, the machine learning model 200 is compact in terms of memory and processing power required, in order to facilitate local training and usage of the machine learning model 200. For example, such a compact model may be a logistic regression model, a linear regression model, or a nonlinear regression model.

At least one processing unit (such as the CPU 12, NPU 13 and/or GPU 30) within the physical activity measurement device 10, or local to the physical activity measurement device 10) operates the machine learning model 200 after that machine learning model 200 is loaded into the memory unit 16. As part of that operation, the at least one processing unit operates the machine learning model 200 during the training of box 306. The training may be performed in real time or near-real-time. As one example, the training of box 306 may be performed in realtime while the user is exercising, during a bout of exercise. As used in this document, the term “realtime” means the actual time during which a thing or an action occurs, and the time within a few seconds after that occurrence. According to other embodiments, the training of box 306 is performed at a discrete time after the user has finished a bout of exercise, where that discrete time is past the point of realtime processing. For example, the at least one processing unit may operate the machine learning model 200 late at night after the user has finished a bout of exercise, because the computational demands on the physical activity measurement device 10 or the smartphone, tablet, smart watch or other user device associated with the physical activity measurement device 10 are typically less at that time.

Next, at box 308, the machine learning model 200 makes one or more useful predictions for the user during a bout of exercise. The machine learning model 200 runs on the at least one processing unit, such as the CPU 12, NPU 13 or GPU 30, which acquires one or more different kinds of data from the sensors described above that transmit data to the at least one processing unit. The machine learning model 200 applies the trained machine learning model to the data that is acquired by the at least one processing unit, and makes one or more useful predictions for the user based on the application of the trained model to the data.

As one example, box 308 may predict the onset of fatigue. The term “bout of exercise,” as used in this document, means a period of time across which a user is engaged in exercise. When a user begins a bout of exercise, there is a strong correlation between the user's physical output (which may be measured in terms of speed, power or other measurement) and the user's heart rate (a measure of how hard the body is working to produce that physical output). At some point during the bout of exercise, the user will begin to fatigue, and that relationship between physical output and the user's heart rate will change. The machine learning model 200 predicts that point of fatigue at box 308, based on data received from the inputs and on the application of the machine learning model 200 to those inputs, and alerts the user to that prediction. The user is alerted through one or more outputs. As one example, a sound may be output from the audio output device 24. The sound may be an alert sound, a vocal output describing the content of the alert (e.g., “fatigue onset is approaching”), or other sound. As another example, visual output such as a message may be displayed on the touchscreen 20, such as “fatigue onset is approaching.” As another example, the touchscreen 20, or a portion thereof, or a light 78, may flash in order to attract the user's attention; such flashing may be performed in conjunction with the display of a message on the touchscreen 20 or by itself. As another example, the mechanical vibration device 26 may output a vibration, such as a long vibration or a vibration pattern. A particular vibration pattern may be associated with a particular type of alert. Optionally, at box 308 the machine learning model 200 may additionally recommend to the user stopping the bout of exercise, or taking a break from the bout of exercise. When tired, a user's exercise form tends to deteriorate, increasing the likelihood of injury or burnout. Such injury or burnout can limit the amount of exercise the user can, or is willing to, engage in, and limit future improvements in fitness and/or training. For highly-motivated athletes who may take the mantra “no pain, no gain” too far, to their detriment, such predictions and recommendations will help them reach their fitness and/or training goals more quickly and in a more healthy manner.

As another example, when a user begins a bout of session, their heart rate will increase from a resting heart rate to a higher active heart rate. That increase in heart rate is a positive for the user, to a point. However, there is a heart rate above which that rate becomes unhealthy for the user. Factors in determining that maximum heart rate include age, gender, altitude, and individual factors. At some point during the bout of exercise, the machine learning model 200 may predict that the user's heart rate may exceed the maximum target heart rate, based on data received from the inputs and on the application of the machine learning model 200 to those inputs, and alerts the user to that prediction. The user is alerted through one or more outputs, as described in the example above. Optionally, at box 308 the machine learning model 200 may additionally recommend to the user stopping the bout of exercise, or taking a break from the bout of exercise.

As another example, a user will need to hydrate throughout the bout of exercise. Common wisdom is that a user should drink water every 20 minutes. However, depending on the physiology of the user and the intensity of the bout of exercise, the user may need to drink water more frequently, or less frequently. Factors in determining when a user should drink water may include heart rate, respiration rate, blood oxygen, the user's body temperature, and blood sugar (glucose), as well as external factors such as ambient temperature, altitude, and humidity. At some point during the bout of exercise, the machine learning model 200 may predict that the user should hydrate at a particular time, based on data received from the inputs and on the application of the machine learning model 200 to those inputs, and alerts the user to that prediction. The user is alerted through one or more outputs, as described in the example above.

As another example, a user may need to consume calories at one or more points during the bout of exercise. Depending on the physiology of the user and the intensity of the bout of exercise, the user may need to consume calories more frequently or consume more calories at a particular time. Factors in determining when a user should consume calories may include heart rate, respiration rate, blood oxygen, and blood sugar (glucose). At some point during the bout of exercise, the machine learning model 200 may predict that the user should consume calories at a particular time, and/or consume a particular amount of calories, based on data received from the inputs and on the application of the machine learning model 200 to those inputs, and alerts the user to that prediction or predictions. The user is alerted through one or more outputs, as described in the example above.

The examples above demonstrate the value of training the machine learning model 200 locally on the user's own data. The trained machine learning model is particularly effective because it has been trained solely on the physiological characteristics of the single user, not on a generic body of data from numerous other people. That is, the machine learning model is individual to and thus customized for the particular user. Each user has a unique physiology. A prediction of user fatigue for a particular individual must be based on that user's own physiology, not on the aggregation of physiologies of numerous random individuals. If the user is overweight and begins an exercise regimen in order to lose weight, that user's fatigue point will be significantly different from a user who is in excellent physical condition and engages in a training regimen to improve their performance in sports such as cycling or running; data collected from the overweight user is essentially irrelevant to the user in excellent physical condition. The use of data that is irrelevant to the user in training the machine learning model 200 would be counterproductive, and lead to errors in prediction that could be avoided easily by training the machine learning model 200 solely on the user's own data. Further, physiological predictions for an individual user correlate to the day-to-day changes in that individual user's fitness and to the particular exercise or training regimen of that individual user (e.g., how many days a week the user performs a bout of exercise, and/or the average duration of the user's bout of exercise). For example, a user may run for a period of time, three days in a row. The first day, the user might take a long time to fatigue, but on the third day, with two days in a row of running in the user's legs, that user will typically fatigue more quickly. As another example, if the user continues a fitness regimen for a long time, that user will become more fit, and the amount of time it may take for the user to fatigue during a run will gradually increase over time.

Optionally, at box 308 the machine learning model 200 may determine that a prediction that it has made has actually occurred. This may occur when the user ignores a prediction that the machine learning model 200 has made, such as a prediction that fatigue onset is imminent. At box 308, upon noting that such a prediction has come to pass, the machine learning model 200 may alert the user that the subject matter of the prediction has occurred, and make another recommendation to the user, such as to cease exercise. The machine learning model 200 may make this recommendation to the user by alerting the user through one or more outputs, such as the touchscreen 20, as described above. The machine learning model that is implemented in the machine learning model 200 may note this occurrence, and use that occurrence as part of the user's physiological data set for continued training.

It is noteworthy that box 308 may continue through the entirety of a bout of exercise, and that the machine learning model that is implemented in the machine learning model 200 may make multiple different predictions at box 308 at the same time or at different times throughout the bout of exercise.

When the user's bout of exercise is complete, the process 300 moves from box 308 back to box 302, and the process 300 repeats for the user's next bout of exercise. Advantageously, each time the process 300 repeats, the box 306 repeats, so that the machine learning model 200 is trained on the user's physiological data set that includes data acquired during the user's most recent bout of exercise. In this way, the machine learning model 200 is constantly up to date on the user's physiology, rather than relying on a static model of the user's physiology. As described above, the machine learning model 200 may train in realtime while the user exercises. In this way, the machine learning model 200 may iterate during a single bout of exercise, such that a prediction made by the machine learning model 200 an hour into a bout of exercise may be different from, and more accurate than, a prediction that would have been made by the machine learning model 200 five minutes into the bout of exercise.

In the specification and claims, references to “a processor” include multiple processors. In some cases, a process that may be performed by “a processor” may be actually performed by multiple processors on the same device or on different devices. For the purposes of this specification and claims, any reference to “a processor” shall include multiple processors, which may be on the same device or different devices, unless expressly specified otherwise.

The subject matter may be embodied as devices, systems, methods, and/or computer program products. Accordingly, some or all of the subject matter may be embodied in hardware and/or in software (including firmware, resident software, micro-code, state machines, gate arrays, etc.) Furthermore, the subject matter may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. By way of example, and not limitation, computer readable media may comprise computer storage media and communication media.

Computer storage media includes volatile and nonvolatile, removable and non-removable media implemented in any method or technology for storage of information such as computer readable instructions, data structures, program modules or other data. Computer storage media includes, but is not limited to, random access memory (RAM), read-only memory (ROM), electrically erasable programmable read-only memory (EEPROM), flash memory or other memory technology, other magnetic storage devices, or any other medium which can be used to store the desired information and which can accessed by an instruction execution system.

When the subject matter is embodied in the general context of computer-executable instructions, the embodiment may comprise program modules, executed by one or more systems, computers, or other devices. Generally, program modules include routines, programs, objects, components, data structures, etc. that perform particular tasks or implement particular abstract data types. Typically, the functionality of the program modules may be combined or distributed as desired in various embodiments.

Additionally, it is understood that any transaction or interaction described as occurring between multiple computers is not limited to multiple distinct hardware platforms, and could all be happening on the same computer. It is understood in the art that a single hardware platform may host multiple distinct and separate server functions.

As used in this document, and as customarily used in the art, terms of approximation, including the words “substantially” and “about,” are defined to mean normal variations in the dimensions and other properties of finished goods that result from manufacturing tolerances and other manufacturing imprecisions, and the normal variations in the measurement of such dimensions and other properties of finished goods.

While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the following claims and their legal equivalents.

What is claimed is:

1. A physical activity measurement device usable by a user, comprising
a processing unit;
at least one input connected to said processing unit, at least one said input related to exercise by the user;
at least one output connected to said processing unit;
at least one storage device; and
a machine learning model stored in said at least one storage device;
wherein said machine learning model utilizes said at least one input to train itself and to make one or more exercise-related predictions to the user.

2. The system of claim 1, wherein at least one input is selected from the group consisting of a touchscreen, a GPS radio transceiver, an accelerometer, a gyroscope, a heart rate sensor, a pulse oximeter, a thermometer, a blood pressure sensor, a respiration rate sensor, a blood lactate sensor, an altimeter, a vibration sensor, a blood glucose sensor, a radar transceiver, a sonar transceiver, a weight sensor, and a clock.

3. The system of claim 1, wherein at least one output is at least one of a touchscreen, a graphical display device, an audio output device, and a mechanical vibration device.

4. The system of claim 1, further comprising at least one item of exercise equipment and one or more inputs connected to said at least one item of exercise equipment.

5. The apparatus of claim 4, wherein at least one input is selected from the group consisting of a torque sensor, an accelerometer, a vibration sensor, a gyroscope, a strain gauge, a force sensor, a velocity sensor, and an angular velocity sensor.

6. A computer-implemented method for machine learning and prediction relating to exercise by a user during a bout of exercise, comprising:
providing a physical activity measurement device that includes at least one input and at least one output;
providing a machine learning model;
collecting physiological data from the user, via the at least one input;
adding said physiological data acquired by said collecting to the user's physiological data set;
training said machine learning model on the user's said physiological data set; and
making at least one prediction for the user based on the application of said machine learning model to at least one input during the bout of exercise.

7. The computer-implemented method of claim 6, further comprising storing said machine learning model on said physical activity measurement device.

8. The computer-implemented method of claim 6, wherein said training is performed locally.

9. The computer-implemented method of claim 6, wherein said training is performed in realtime during a bout of exercise.

10. The computer-implemented method of claim 6, wherein said making is performed locally.

11. The computer-implemented method of claim 6, further comprising communicating said at least one prediction to the user via said at least one output.

12. The computer-implemented method of claim 6, wherein said making at least one prediction for the user comprises making a plurality of predictions for the user during a bout of exercise.

13. The computer-implemented method of claim 6, further comprising, before said making a prediction, determining whether said physiological data set is sufficiently large to allow said machine language model to perform said making a prediction.

14. The computer-implemented method of claim 6, wherein said training is performed on said physical activity measurement device.

15. The computer-implemented method of claim 6, wherein said training occurs in realtime during said adding.

16. The computer-implemented method of claim 8, wherein said training occurs at a discrete time after said adding.

17. A computer-readable medium that configures a processing unit of a user's physical activity measurement device, which includes at least one input and at least one output in communication with the processing unit, to perform:
collecting physiological data from the user, via the at least one input;
adding said physiological data acquired by said collecting to a user's physiological data set;
training a machine learning model on the user's said physiological data set; and
making at least one prediction for the user based on the application of said machine learning model to at least one input during the bout of exercise.

18. The computer-readable medium of claim 17, further comprising communicating said at least one prediction to the user via said at least one output.

19. The computer-readable medium of claim 17, wherein the computer-readable medium is a storage device in the physical activity measurement device.

20. The computer-readable medium of claim 17, wherein said training and said making are performed locally.
